(19) United States
(12) Patent Application Publication
(10) Pub. No.: US 2011/0066450 A1
(43) Pub. Date: Mar. 17, 2011

(54) System for providing bio-identical hormone replacement therapy

(76) Inventor: Cynthia Karapanos, Webster, MA (US)

(21) Appl. No.: 12/586,117

(22) Filed: Sep. 17, 2009

(51) Int. Cl. G06Q 50/00; A61B 5/00

(52) U.S. Cl. 705/3

(57) ABSTRACT

A single stop computer-driven system is provided within a retail pharmacy to evaluate, test and formulate regimens for the treatment of PMS, menopause, and other illnesses relating to hormone imbalances.

### FIELD OF THE INVENTION

This invention relates to a system for providing bio-identical hormone replacement therapy and more particularly to a system for generating a customized prescription based on evaluation and testing within a retail pharmacy.

### BACKGROUND OF THE INVENTION

Prescription solutions to premenstrual syndrome or PMS and menopause are marketed and sold through customized prescriptions for various types of hormones in which prescriptions are written at a doctor's office. However the above therapies depend on a doctor who is knowledgeable in treatment of PMS and menopause for which many general practitioners are ill-equipped and uneducated. Thus, present systems for disseminating health care and more particularly for PMS and menopause problems is uniquely dependant on a doctor-focused approach to healthcare. When one is sick, one has to schedule an appointment with one's doctor. But the problem with going to a doctor for help with PMS or menopause is that doctors are not exposed to PMS and menopause treatment in medical school. In point of fact, for a long time PMS and menopause were not considered illnesses. Therefore most doctors do not know how to identify, diagnose or treat PMS or menopause.

The second problem having to do with the treatment of PMS and menopause is the limited ability to create awareness. Due to the nature of pharmaceutical sales it is virtually impossible to educate enough doctors to treat PMS and menopause because it is difficult to get face-to-face meetings with doctors. Even if detail people responsible for promoting PMS and menopause treatments could get in front of a doctor, and assuming the doctor is willing to be educated, there is still a problem of the PMS patient finding a PMS doctor.

Thus it is very difficult for a woman having PMS or menopause problems to find a doctor to treat PMS or to find a solution on her own. It has been said there is a higher probability of being hit by lightning than finding a PMS or menopause specialist.

Patient struggles with PMS and menopause are legendary, with the symptoms including extreme irritability, mood swings and mental confusion. While PMS was identified in the 1940's, treatment options were unavailable in the United Sates until the late 1970's. Moreover, these treatment possibilities were only available from compounding pharmacies generally not well known to the medical community or the public at large.

Thus, in terms of patient struggles, heretofore the patient has been forced to find a solution on her own. Unfortunately this process is just as frustrating and confusing as relying on doctors who are ill-equipped to treat PMS. Most of the time there is no local access to experts and the paralyzing symptoms of PMS, such as mental confusion and depression, make the entire process of diagnosis and obtaining a cure difficult.

While hormone replacement therapy has been available in terms of synthetic hormones, it was found that these synthetic hormones are sufficiently different from body-produced hormones that they can in fact cause disease and are therefore counter-indicated. This has led to the development of bio-identical hormone replacement therapy which is now considered safe and effective. Note that until 2002 the most popular hormone replacement therapy drugs were Premarin which only contains synthetic estrogen, as well as Provera which contains synthetic progesterone and Prompro which is a combination of the synthetic estrogens and progesterone.

It is therefore a requirement that the PMS or menopause patient be made aware of bio-identical hormone replacement therapy and be provided with prescriptions to ensure that the bio-identical hormone replacement medications are specifically tailored to her.

In the past, and as shown in U.S. Pat. No. 6,174,665, either blood tests or saliva tests have been used to derive a female hormone profile utilized in the formulation of the therapy, with blood tests requiring a nurse. As for saliva testing, in one instance a comprehensive salivary assessment of estradiol, progesterone and testosterone spanning a full 28-day period is used to generate a bio-identical hormone replacement regime. Imbalances revealed in this profile were used to illuminate root causes of disorders such as premenstrual syndrome, infertility and menstrual irregularities.

The problem with the generation of a female hormone profile in this manner is that it requires providing 11 saliva samples over the full 28-day period to establish levels of estradiol and progesterone. Testosterone is measured from the 28^{th} day specimen, with the levels of estradiol of progesterone as well as the ratio between the two plotted on a graph enabling the practitioner observe levels throughout the month, as well as the relationship between the two hormones. It is said that imbalances such as unopposed estrogen, high follicular progesterone, anovulation, and luteal phase defects can be identified, providing clues about the contributing factors to irregular cycles, infertility, PMS and other chronic gynecological disorders.

Salivary hormone analysis offers a distinct advantage over blood-test derived plasma measurements that typically reflect both bound and unbound fractions of hormones. This is because salivary samples represent the free or unbound bio-available fraction of hormone. Because various factors such as such as obesity and thyroid function influence levels of sex hormone-binding globulin, salivation analysis can identify a functional deficiency or excess not apparent in plasma measurement.

As can be seen from the above, first it is important to be able to have the patient be aware of the therapies available for PMS and menstrual problems. This is followed by the necessity of a knowledgeable doctor. Third is the location of a compounding pharmacy to be able to provide the appropriate therapy. Note that it is the purpose of the compounding pharmacies to customize dosage and delivery systems.

Add to this the problem of in-home testing over long periods of time that militates against the ability to easily obtain relief for PMS and menopause symptoms. As will be appreciated, prolonged in home testing regimens are not likely to be continued or properly administered.

In summary, none of the above treatments are available outside a doctor's office.

### SUMMARY OF INVENTION

A single stop computer-driven system is provided within a retail pharmacy to evaluate, test and formulate regimens for the treatment of PMS and menopause. For this purpose an area of the retail pharmacy is blocked off or dedicated to provide a specialized clinic within the retail pharmacy. The clinic offers seminars in PMS and menopausal problems to inform patients about the problem and available therapies and to market and sell bio-identical hormone replacement therapies. This area within the retail pharmacy offers saliva testing as opposed to blood testing that does not require licensed nurses to perform the test.

Concomitant with the saliva testing the results of which are uploaded to a local computer is an evaluation which is likewise input to the computer so that an appropriate profile can be generated at the retail pharmacy-based clinic. This results in the entire procedure being done at the retail clinic, and in general in a single visit. The clinic provides the aforementioned seminar, evaluation of health histories, the performance of the salivation test, computer-based lab interpretation of the test results and a computer-generated custom prescription which is shipped out to a compounding pharmacy if one is not available at the retail pharmacy.

It is one of the features of the subject invention, it has been found that a single salivary test is effective to enable compounding of a prescription, making the treatment of PMS and menopause available through one visit to the clinic within the retail pharmacy.

The retail pharmacy provides the ability for an individual to know that help is available within the confines of the clinic. The in-store publicity includes the informative seminars which provide women with the knowledge that traditional PMS and menopause problems in fact do have a solution. The clinic advertises the availability of such a service within the retail pharmacy by signs or advertisements within the retail pharmacy, such that treatment options never available outside a doctor's office are now available in a single visit to the retail pharmacy.

Thus, in order to solve the problem of the lack of awareness of the availability of prescription solutions for PMS and menopause problems, retail pharmacy advertising of on-site help eliminates the problem of finding a knowledgeable doctor and combats the limited awareness by patients and doctors as to the solutions to PMS and menopause symptoms.

In the subject system only bio-identical hormone therapies are available. This eliminates the counter-indications of the synthetic hormone replacement therapies such as breast cancer, strokes, blood clots and increases in the instances of cardiovascular disease.

More particularly, the focus of the subject invention and the most important component in the process is creating and maximizing awareness for as many women as possible. This is accomplished by advertising and offering educational seminars at the retail pharmacy clinics, with the retail pharmacy clinics being conveniently located in various cities across the country.

In one embodiment, seminars are held at regular times during the day and are advertised on the marquees at select locations. This type of exposure combined with local access to educational seminars and a hormone balance specialist gives women the opportunity to make informed and therapeutic decisions. The subject system makes finding and getting help for PMS and menopause easy for as many women as possible, while at the same time attracting patients to the clinics.

The first step in the process is to evaluate health histories and hormone levels. A patients symptoms' are assessed at the clinic and entered into a computer. Next lab samples are collected and provided to a diagnostic testing lab, also on premises, that has expertise in testing and analyzing steroid hormones.

The second step of the process is for the computer to interpret lab results and to make recommendations for hormone therapy. The computer employs an expert system that in effect provides hormone balance specialists within the clinics to review hormone profiles and suggest treatment options to assist the patient in determining the best course of action.

The third step is to enter the appropriate hormone therapy into the computer in which the appropriate hormone therapy is based on an assessment of the patients symptoms, lab results and recommended treatment options.

A customized prescription is then created by the computer at the clinic and is transmitted to a compounding pharmacy, whether the compounding pharmacy is within the retail pharmacy or at some remote location.

The customized prescription therapy involves formulation by a compounding pharmacy, with the resultant formulation delivered back to the retail clinic, is compounded on the spot, or is sent out to the patient.

In one embodiment, the computer prompts at the retail pharmacy to suggest follow up testing conducted over a series of months, for instance in the first year, and then once a year thereafter. This is to assure the prescription delivers replacement hormones in exactly the amount needed, and to help avoid the side effects with overdosing or under-dosing. This type of computer-controlled ongoing case management and monitoring of outcomes allows the patient to continue to evaluate and adjust their plan of care to attain optimal therapeutic outcomes or to involve hormone therapy replacement specialists at the clinic within the retail pharmacy to assist.

Note a custom compound is defined as a prescription made by a pharmacist from scratch using raw materials according to a prescription written by a doctor.

In summary, a system is provided to provide PMS and menopause information and awareness, diagnosis and compounding pharmacy prescriptions all within a single retail pharmacy involving a single patient visit such that bio-identical hormone replacement therapies can be formulated and filled at the pharmacy or sent out to a compounding pharmacy.

While the subject invention has been described in terms of obtaining medical assistance for PMS and menopause, it will be appreciated that a one stop, one visit approach to providing formulations to address other hormonal imbalance related issues is within the scope of the subject invention.

For instance, hormone imbalance, when measured, can result in forms that combat aging, the effects of a hysterectomy, reproductive health issues, vaginal health problems, and reduced libido. Also, while the subject invention is described to address female concerns, male concerns including andropause can also be addressed in the above manner.

Thus, for instance, formulations provided by the subject invention can alleviate the following symptoms: allergies, anxiety, breast swelling, breast tenderness, cold body temperature, depression, drowsiness, fatigue, feel burned out, feel pressured for time, feel ‘tired but wired’, foggy thinking, hot flashes, irritability, memory lapses, mood swings, morning sluggishness, muscle aches/stiffness, night sweats, poor exercise tolerance, sleep disturbances, tearfulness, water retention, weight gain-waist.

### BRIEF DESCRIPTION OF THE DRAWINGS

These and other features of the subject invention will be better understood in connection with the Detailed Description, in conjunction with the Drawings, of which:

FIG. 1 is a diagrammatic illustration of the utilization of a PMS and menopause center within the confines of a retail pharmacy to provide PMS and menopause therapies in a one stop shopping, one visit scenario;

FIG. 2 is a diagrammatic illustration of the functions carried out in the clinic of FIG. 1 showing an initial sign up, seminars, evaluation, saliva testing, lab interpretation and the generation of a custom prescription; and,

FIG. 3 is a block diagram of the computerized system that implements the subject system illustrating the utilization of a processor having multiple inputs including saliva testing results and patient information to be able to generate a custom prescription which is linked to a compounding pharmacy, whether the compounding is within the retail pharmacy or at a remote location;

### DETAILED DESCRIPTION

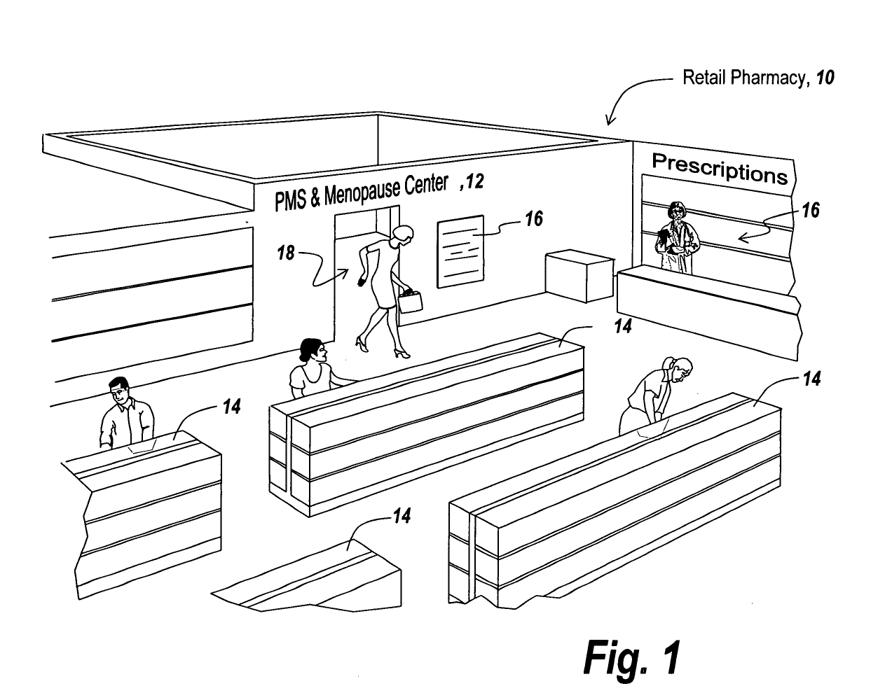

Referring to FIG. 1, a retail pharmacy 10 is provided with a PMS and menopause center or clinic 12 which is shown in the corner of the retail pharmacy and which occupies its own space. Here it can be seen that the remainder of the pharmacy has the traditional rack space 14 and a prescription counter 16 adjacent in one embodiment to the PMS menopause center.

It is the purpose of the PMS menopause center to provide one stop, one visit shopping for individuals in need of such treatment, with the PMS and menopause center having advertising or indicia at the clinic to indicate the presence of the PMS and menopause center as well as PMS and menopause treatment and awareness displays 16, for instance adjacent the entrance or portal 18 of the center or clinic 12.

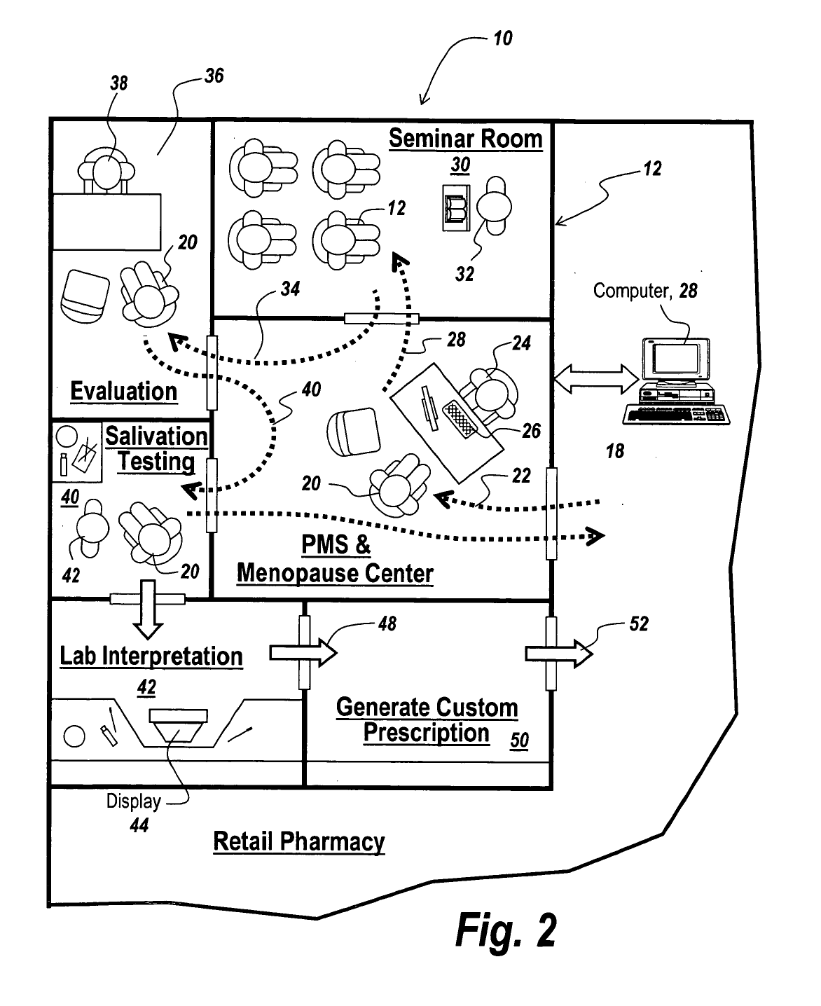

Referring now to FIG. 2, an individual interested in obtaining PMS and menopause treatment, here illustrated at 20, enters portal 18 as illustrated by dotted line 22 where the initial consult is set up by an individual 24 having a keyboard entry device 26 coupled to a computer 28.

After the initial consult, as illustrated by dotted arrow 28, the individual 20 goes to a seminar room or area 30 which is part of center 12 at which point awareness of treatment options are discussed by a seminar presenter 32.

After the seminar, as illustrated by dotted line 34, individual 20 is provided with evaluation at an evaluation area 36 which is staffed by an individual 38 to obtain vital information about the patient history and patient condition.

Thereafter as illustrated by a dotted arrow 40, the patient 20 goes to a salivation testing area 40 at which a saliva sample is taken by an individual 42 noting the date and time. The sample is either analyzed at the retail pharmacy or is sent out to a specialized laboratory. The saliva testing results are then inputted into computer system 28.

The results of the salivation testing are made available to a display 44 in lab interpretation area 42 in which the results are analyzed. The analysis in one embodiment is done by computer 28, with the result as illustrated by arrow 48 being the generation of a custom prescription 50 which is transmitted as illustrated by arrow 52 to a compounding pharmacy, in one embodiment not located within the retail pharmacy 10, but rather at a remote site.

Alternatively, for some retail pharmacies the compounding may be done within the retail pharmacy itself.

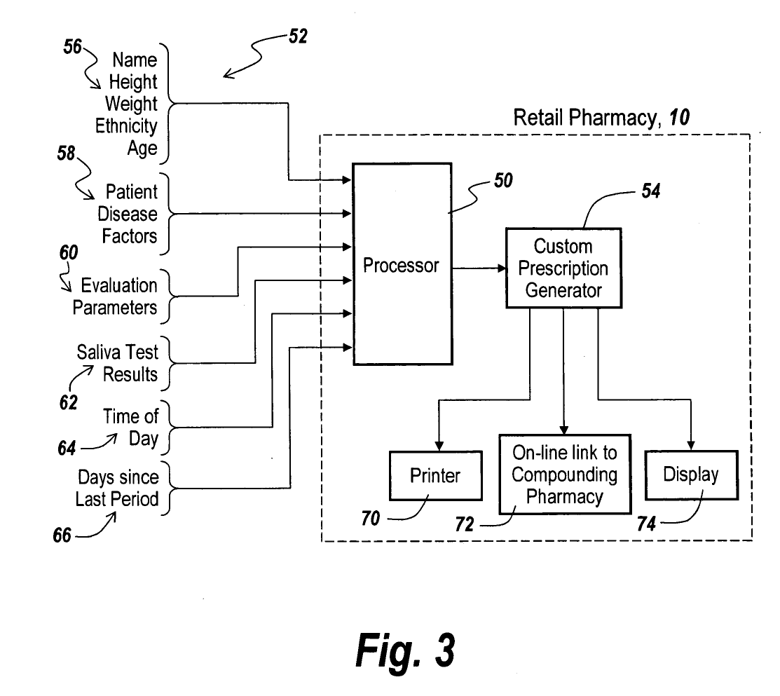

Referring now to FIG. 3 in terms of the computer-assisted system, retail pharmacy 10 is provided with computer 28, in this case processor 50 which is provided with inputs generally illustrated at 52 to enable the assessment of a patient and the generation of a custom prescription as illustrated at 54.

It is a finding of the subject invention that the physical parameters of the patient are input to processor 50 including the name, height, weight, ethnicity, age and other factors, as illustrated at 56, and assuming patient disease factors such as diabetes and the like are input as illustrated at 58, then given certain evaluation parameters 60 relative to the female hormone profile required and the salivation test results 62, then processor 50 is programmed provided a formulation in terms of a custom prescription for the individual, given the fact that the time of day 64 is entered into processor 50. For PMS, the days since the individuals' last period 66 are also entered into processor 50.

The custom prescription 54 is coupled to a printer 70, an online link to a compounding pharmacy 72 and a display 74 such that the result of the single test and single visit is available to the compounding pharmacy.

It is the purpose of the subject system to generate salivary estradiol and progesterone activity report which is correlated to the day of the month for the woman involved as well as the time of day. It has been found that given certain patient parameters that have been entered into the system, a single saliva test can provide sufficient information to be able to formulate a prescription for the PMS or menopause problem involved.

As further inputs to the system, woman's menstrual cycle levels of estradiol, progesterone and testosterone may be coupled with information relating to mood, sleep patterns, appetite and sexual drive to arrive at an appropriately balanced hormone replacement therapy.

It is noted that chronic imbalances of the above hormones are implicated in disorders such as PMS and menopause. However, while it has been previously thought that a single measurement such as done with a single serum measurement of sex hormones was inadequate to be able to specify a hormone replacement regime, it has been found that a single test at a single point in time makes possible a single visit to the retail pharmacy in order to provide the relief required.

Various compounding pharmacies such as for instance Madison Pharmacy Associates are utilized in compounding formulations for treatment of hormone replacement therapy.

These laboratories evaluate estradiol, progesterone, the progesterone to estradiol ratio, testosterone, cortisol and DHEA deficiencies.

What is now presented are some of the factors that are taken into consideration by the compounding laboratory which enables the compounding laboratory to provide the appropriate formulation for the patient. All of the data required for lab interpretation is available from computer 28.

### Low Hormone Levels

Critical considerations for low hormone levels with respect to estradiol indicate nutritional supplements including boron. Also involved is progesterone supplementation which may facilitate estradiol receptor signaling and improve the clinical effects of E2.

Also with respect to low estradiol levels is estrogen supplementation in order to maintain balance. Note progesterone should always accompany estrogen supplementation, regardless of estrogen type, dose, route of administration or whether the uterus is intact.

The subject system also ascertains the minimum dosage to be required to relieve symptoms. Also, as part of the low estradiol levels lifestyle changes may be an important consideration such as weight gain if the patient is currently underweight, whereas periodic monitoring of bone density in the face of low E2 may be advisable.

With respect to low levels of progesterone, considerations of nutritional supplements include vitex/chaste berry action on dopamine receptors and decreases in prolactin which may help normalize luteal phase progesterone.

There is also progesterone supplementation for low levels which may be accomplished through progesterone cream, oral micronized progesterone or compounded sustained released micronized progesterone. It is noted that the minimum dose required to relieve symptoms is a consideration. Moreover, with women on birth control pills, the benefit from progesterone cream supplementation may be evaluated. The final low progesterone consideration is the optimization of thyroid function where low Pg may be symptomatic of low T3, as ovaries need T3 to secrete adequate progesterone. The progesterone supplementation can help to optimize the thyroid function.

With respect to low testosterone and there is of course the consideration of testosterone supplementation, for instance by transdermal testosterone, with doses commonly ranging from 0.25 to 2 mg/DA. Oral testosterone may be administered utilizing testosterone undeconate used in one capsule every other day. Co-ingestion of 20 grams of fat in some instances helps decrease first pass metabolism. Also as a testosterone consideration is the periodic monitoring of bone density in women with low testosterone.

With respect to low DHEA-S, nutritional supplements should be considered with an adrenal support protocol particularly if low cortisol is also present. DHEA supplementation may be indicated through oral or sublingual administration, with a minimum dose required to be ascertained to relieve symptoms and stay within range. It is noted also that low DHEA may be associated with hypothyroidism, chronic stress and/or chronic illness.

Finally as to low cortisol, the considerations are to assess diurnal cortisol via Adrenal Function Panel or by a 4 point cortisol saliva test. Nutritional supplements include an adrenal support protocol, with cortisol supplementation including short term supplementation which may be required in some cases. Finally sleep assessment may be critical in the low morning cortisol associated with insomnia.

### High Hormone Levels

As to critical considerations for high levels of estradiol, prolonged excessive estrogen supplementation can lead to down regulation of estrogen receptors and may result in deficiency symptoms even though measured levels are high. Also with respect to estradiol, a consideration is to reduce the dose of estradiol when salivation estradiol levels are high and optimal clinical results have not been achieved. Moreover, when saliva E2 levels are above or at the high end of the range, symptoms of excess breast tenderness, migraines, fluid retention and weight gain at the hips are present.

Estradiol (E3) is an end metabolite and bioaccumulation may occur. If the clinical response is good, monitoring of E3 levels more frequently is desirable. If the levels remain high and symptoms worsen one would consider reducing the E3 dose.

If the patient is clinically stable for greater than 6 months and no E2 excess symptoms or side effects occur one could consider monitoring E2 more frequently.

One could also reduce estrogen synthesis through weight loss and one could use progesterone supplementation to balance the effects of estrogen. As to nutritional supplements, vitamin B3 is a cofactor in estrogen metabolism and may be used to improve liver and bowel function to assist in estrogen elimination. Also, it is desirable to check high thyroid hormone levels since high estrogens can interfere with tissue action of T3.

With respect to high progesterone levels, prolonged excessive progesterone supplementation can lead to down regulation of progesterone and estradiol receptors and possible deficient symptoms even if measured levels are high. In addition, high endogenous progesterone is generally not problematic, whereas one may wish to reduce the dose of progesterone when saliva Pg levels are above or at the high end of the range and symptoms of excess are present including breast swelling, depression and excessive drowsiness. Nutritional supplements may be added such as omega 3 acid supplementation to prevent accumulation of testosterone.

With respect to high levels of testosterone, prolonged excessive testosterone supplementation can lead to down regulation of testosterone receptors and may result in deficiency systems even though measured levels are high. The indicated response is to reduce the dose of testosterone when saliva testosterone levels are at the high range and symptoms of excess are present including hirsutism, oily skin and acne.

Moreover as to high levels of progesterone, progesterone cream may cause elevation of salivary testosterone due to conversion of progesterone and androstenedione and testosterone. Moreover if the patient is on oral Pg and testosterone is high, one should consider switching to progesterone cream. Also, investigating polycystic ovary syndrome (PCOS) and/or insulin resistance is recommended as these conditions have a strong association with elevated androgens.

As to high levels of DHEA-S, one would consider reducing the dose of DHEA to small doses less than 10 milligrams. These doses are generally effective in women. Finally, one should evaluate weight loss in which weight loss results in elevated DHEA-S associated with central adipo site. As to nutritional supplements, adrenal support protocol may be called for high DHEA and one could consider investigating for PCOS and/or insulin resistance due to a strong link to excess androgens. Finally, one might wish to institute measures to reduce insulin resistance inducing weight loss and exercise.

Finally with respect to high levels of cortisol, it is important to assess diurnal cortisol via Adrenal Function Panel or the 4 point cortisol saliva test. High cortisol may be resolved with nutritional supplements including an adrenal support protocol. Moreover, it is important to assess sleep since difficulty sleeping may be the result of high bedtime cortisol. As to weight loss, elevated cortisol is associated with weight gain, whereas it is in generally important to test for metabolic syndrome if necessary.

It will be appreciated that in the formulation of a prescription the progesterone-estradiol ratio should be evaluated as well as the progesterone-estradiol balance. In terms of prescriptions involving progesterone cream, it is noted that the salivary progesterone level after topical progesterone application is mostly qualitative in nature. Nevertheless there is value in knowing whether the level is above or below range. If the progesterone level is unexpectedly low and clinical response is lacking perhaps a different carrier in terms of cream or base may be needed. If suboptimal clinical results are seen it may indicate that the progesterone level is below range, whereas if the progesterone level is above range it may be an indicated of excess supplementation, with symptoms including drowsiness and breast swelling.

In short, all of the above factors are utilized in the preparation of a formulation to combat PMS and menopause symptoms, and all of these factors can be ascertained from one saliva test along with associated data.

In summary, the utilization of a PMS and menopause center within the retail pharmacy provides easy access to women in a one stop, one test process so that they can be made aware of the treatment options and be provided with a prescription tailored to their own needs.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A computer-driven system for generating a customized prescription based on evaluation and testing within a retail pharmacy, comprising:
a retail pharmacy;
a blocked off area within said retail pharmacy for housing a PMS/menopause center;
a seminar area within said center for providing information relating to PMS/menopause treatment for a patient;
an evaluation area for taking down a history of a patient and entering said patient history into a computer;
a salivation testing area for taking a saliva sample from said patient and for entering the result thereof into said computer; and,
a lab interpretation area having a processor coupled to said computer for downloading the results of said salivation testing and for interpreting said results for the purpose of generating a prescription, said computer being provided with said lab interpretation and generating a custom prescription based thereon, whereby a single stop, single visit to a PMS and menopause center within said retail pharmacy results in the generation of a custom prescription to be transmitted to a compounding pharmacy for formulation.

2. The system of claim 1, wherein said computer is provided with the name, height, weight, ethnicity and age of said patient.

3. The system of claim 2, wherein said computer is provided with patient disease factors.

4. The system of claim 3, wherein said computer is provided with parameters useful in the evaluation of PMS or menopause conditions.

5. The system of claim 2, wherein said computer is provided with results of a saliva test, the time of day of said saliva test and the days since the onset of the last period of said patient.

6. The system of claim 1, wherein said computer is provided with an expert system for analyzing the inputs thereto and for generating a custom prescription relating to the treatment of PMS or menopause symptoms.

7. A system for providing one stop shopping for a patient suffering from the symptoms of PMS or menopause, comprising:
a PMS/menopause center within a retail pharmacy for providing awareness for PMS or menopause treatment options, for patient evaluation, for providing a single saliva test, for the analysis of said saliva test including interpretation thereof, and for generating a custom prescription for a formulation to treat the PMS or menopause symptoms of said patient.

8. The system of claim 7, wherein said formulation is sent to a compounding pharmacy for fulfillment.

9. The system of claim 8, wherein said compounding pharmacy is remote from said retail pharmacy.

10. The system of claim 7, wherein said compounding pharmacy is within said retail pharmacy.

11. The system of claim 7, wherein a single saliva test is sufficient to permit generation of said formulation, whereby a patient in a single visit to a single location can be made aware of treatments for PMS or menopause and be provided with a prescription for a formulation for the treatment of PMS or menopause.

12. The system of claim 7, wherein said evaluation, said saliva testing and said prescription generation are computer-assisted.

13. The system of claim 12, wherein the computer utilized for said computer assistance is provided with the results of said saliva test, the time of day of said saliva test and the days since the onset of the last period of said patient.

14. The system of claim 12, and further including a display for displaying the results of said formulation.

15. The system of claim 12, and further including an online link from said computer to a compounding pharmacy.

16. The system of claim 15, wherein said compounding pharmacy is remote from said retail pharmacy.

17. The system of claim 7, wherein said saliva test provides a female hormone profile.

18. The system of claim 17, wherein said hormone profile includes salivary estradiol and progesterone levels.

19. A computer-driven system for generating a customized prescription based on evaluation and testing within a retail pharmacy, comprising:
a retail pharmacy;
a blocked off area within said retail pharmacy for housing a hormone imblance center;
a seminar area within said center for providing information relating to PMS/menopause treatment for a patient;
an evaluation area for taking down a history of a patient and entering said patient history into a computer;
a salivation testing area for taking a saliva sample from said patient and for entering the result thereof into said computer; and,
a lab interpretation area having a processor coupled to said computer for downloading the results of said salivation testing and for interpreting said results for the purpose of generating a prescription, said computer being provided with said lab interpretation and generating a custom prescription based thereon, whereby a single stop, single visit to a hormone imbalance center within said retail pharmacy results in the generation of a custom prescription to be transmitted to a compounding pharmacy for formulation.

20. The system of claim 19, wherein said computer is provided with the name, height, weight, ethnicity and age of said patient.

21. The system of claim 20, wherein said computer is provided with patient disease factors.

22. The system of claim 21, wherein said computer is provided with parameters useful in the evaluation of hormone imbalances.

23. The system of claim 20, wherein said computer is provided with results of a saliva test, the time of day of said saliva test.

24. The system of claim 19, wherein said computer is provided with an expert system for analyzing the inputs thereto and for generating a custom prescription relating to the treatment of hormone imbalance symptoms.

25. A system for providing one stop shopping for a patient suffering from hormone imbalance symptoms, comprising:
a hormone imbalance center within a retail pharmacy for providing awareness of hormone imbalance treatment options, for patient evaluation, for providing a single saliva test, for the analysis of said saliva test including interpretation thereof, and for generating a custom prescription for a formulation to treat the hormone imbalance symptoms of said patient.
